(19) United States
(12) Patent Application Publication
(10) Pub. No.: US 2009/0232897 A1
(43) Pub. Date: Sep. 17, 2009

(54) PHARMACEUTICAL COMPOSITIONS COMPRISING CONJUGATED ESTROGENS

(76) Inventors: Bijayananda Sahoo, Bhadrak (IN); Pratit Premchand Agrawal, Gondia (IN); S.H. Seyed Mohamed Buhary, Tirunelveli (IN); Pradip Kumar Ghosh, Midnapur West (IN); Anand Sankaranarayanan, Chennai (IN); Subhash Gore, Sholapur (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/403,401

(22) Filed: Mar. 13, 2009

Related U.S. Application Data
(60) Provisional application No. 61/048,573, filed on Apr. 29, 2008.

(30) Foreign Application Priority Data
Mar. 14, 2008 (IN) 642/CHE/2008

(51) Int. Cl. A61K 9/14; A61K 31/56

(52) U.S. Cl. 424/490; 514/182

(57) ABSTRACT

Pre-mix compositions containing conjugated estrogens and a pharmaceutical carrier, and pharmaceutical formulations containing a pre-mix composition. Further, the invention includes processes for preparing the premix compositions and pharmaceutical formulations containing pre-mix compositions.

An aspect of the present invention relates to pre-mix compositions comprising conjugated estrogens and at least one pharmaceutically acceptable carrier, and processes for the preparation of the pre-mix compositions.

Another aspect of the invention relates to pharmaceutical formulations comprising conjugated estrogens pre-mix compositions and processes for preparing the same. The invention also pertains to therapeutic uses and methods of treatment employing pre-mix compositions comprising conjugated estrogens or their pharmaceutical formulations.

Conjugated estrogens comprise a mixture of sodium salts of water-soluble estrogen sulfates, representing the average composition of steroid material derived from pregnant mare urine. It is primarily a mixture of sodium estrone sulfate (1) and sodium equilin sulfate (2). It also contains additional minor components as concomitant components, including: sodium 17α-dihydroequilin sulfate (3); sodium 17α-estradiol sulfate (4); sodium 17β-dihydroequilin sulfate (5); sodium 17α-dihydroequillenin sulfate (6); sodium 17β-dihydroequilenin sulfate (7); sodium equilenin sulfate (8); sodium 17β-estradiol sulfate (9); and sodium Δ^{8,9}-dehyroestrone sulfate (10); a structural formula for each of these named components being shown below.

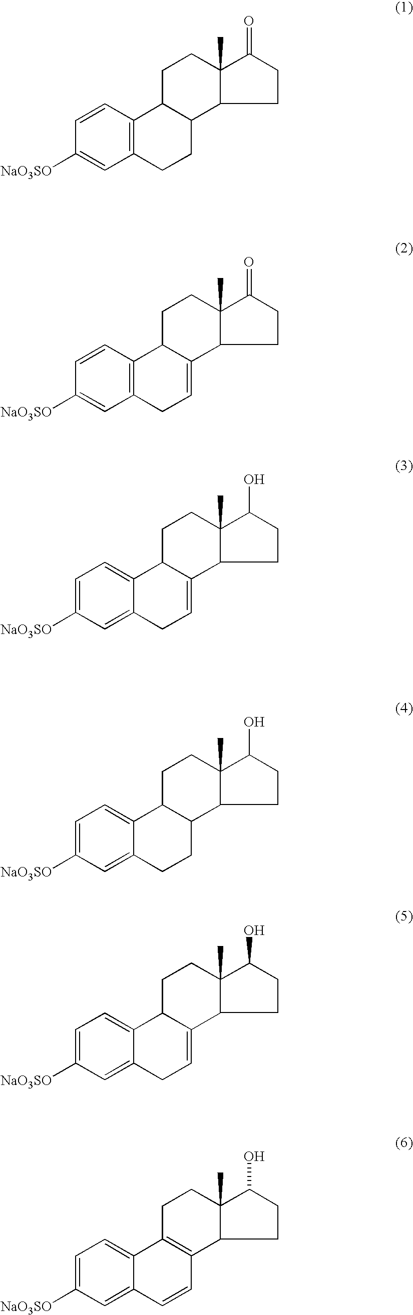

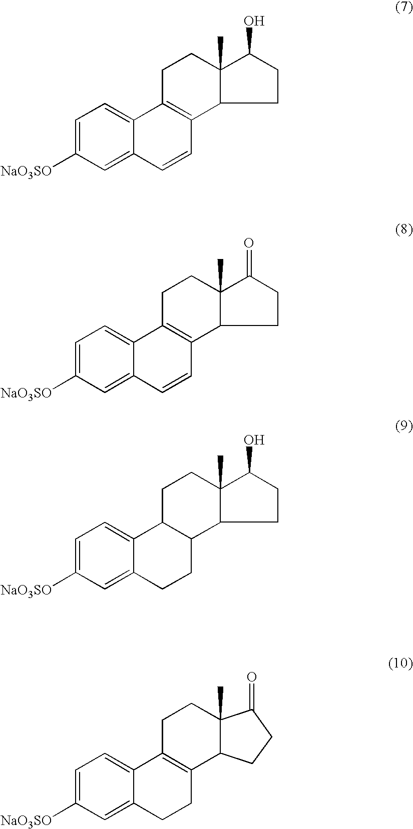

The monograph for Conjugated Estrogens in United States Pharmacopoeia 29, United States Pharmacopoeial Convention, Inc., Rockville, Md., 2005 (“USP”) at pages 849-850 specifies the following component concentrations, based on the labeled conjugated estrogen content:

1) Not less than 52.5% and not more than 61.5% of sodium estrone sulfate;

2) Not less than 22.5% and not more than 30.5% of sodium equilin sulfate;

3) A total of sodium estrone sulfate and sodium equilin sulfate not less than 79.5% and not more than 88.0% of the labeled conjugated estrogen content; and

4) Concomitant components, as sodium sulfate conjugates:
a) Not less than 13.5% and not more than 19.5% of 17α-dihydroequilin;
b) Not less than 2.5% and not more than 9.5% of 17α-estradiol; and
c) Not less than 0.5% and not more than 4.0% of 17β-dihydroequilin.
   This USP monograph also sets upper limits for the concentrations of some other estrogen components.

The USP monograph for Conjugated Estrogens Tablets, at pages 851-852, specifies that the tablets will contain not less than 73.0% and not more than 95.0% of the label amount of conjugated estrogens, as the total of sodium estrone sulfate and sodium equilin sulfate. The ratio of sodium equilin sulfate to sodium estrone sulfate is to be not less than 0.35 and not more than 0.65.

Conjugated estrogens are currently available in products sold as PREMARIN® tablets for oral administration, available in strengths of 0.3 mg, 0.45 mg, 0.625 mg, 0.9 mg, and 1.25 mg of conjugated estrogens, and formulated with the following excipients: calcium phosphate tribasic, hydroxypropyl cellulose, microcrystalline cellulose, powdered cellulose, hypromellose, lactose monohydrate, magnesium stearate, polyethylene glycol, sucrose, and titanium dioxide.

Conjugated estrogens have been used for many years as estrogen supplements in order to treat or prevent a variety of conditions that are induced or exacerbated by estrogen hormone deficiency. Particularly, conditions experienced by pre-menopausal, menopausal, and post-menopausal women such as osteoporosis, hot flashes, vaginal atrophy, and loss of protection against heart attacks, can be ameliorated using conjugated estrogens as part of an estrogen replacement therapy.

International Application Publication Nos. WO 98/08525 and WO 98/08526 disclose processes to obtain an extract containing a natural mixture of conjugated estrogens from mare's urine by solid-phase extraction, and by using non-ionic semipolar polymer adsorbing resins. However, certain unavoidable fluctuations are always associated with solution extracts of conjugated estrogens due to their origin, storage, transport and pre processing practices.

U.S. Pat. Nos. 5,908,638 and 6,630,166, and U.S. Patent Application Publication Nos. 2004/0131683 and 2005/0271724, disclose compositions comprising conjugated estrogens.

U.S. Patent Application Publication Nos. 2005/0009800 and 2005/0019408 disclose pre-formulations in the form of solid free-flowing dry extracts of natural conjugated estrogens.

The literature teaches that administered estrogens and their esters are processed within the body essentially the same as endogenous hormones. Metabolic conversions of estrogens occur primarily in the liver, but also at local target tissue sites. Complex metabolic processes result in a dynamic equilibrium of circulating conjugated and unconjugated estrogenic forms, which are continually interconverted, especially between estrone and estradiol and between esterified and non-esterified forms. A significant proportion of the circulating estrogens exist as sulfate conjugates, especially estrone sulfate, which serves as a circulating reservoir for the formation of more active estrogenic species.

As conjugated estrogens are complex mixtures of many components, they are prone to inter conversion reactions of some of the constituents, and preparing stable pre-mix compositions and pharmaceutical formulations is a difficult task for the formulation scientist.

Therefore, a need exists for pharmaceutical formulations comprising conjugated estrogens, which have high content uniformity and stability.

### SUMMARY

The present invention relates to pre-mix compositions comprising conjugated estrogens and at least one pharmaceutical carrier.

Further, the invention relates to processes for preparing pre-mix compositions comprising conjugated estrogens and at least one pharmaceutical excipient.

In an embodiment, the invention relates to spray drying processes for preparing pre-mix compositions comprising conjugated estrogens and at least one pharmaceutical carrier, wherein an embodiment of a process comprises:

1) Dissolving or dispersing drug in a suitable solvent. 2) Dissolving or dispersing a suitable carrier in the solution or dispersion of 1). 3) Spray drying the mixture from 2) to evaporate solvent. 4) Optionally, drying the solid obtained from 3).

In an embodiment, the invention includes pharmaceutical formulations comprising conjugated estrogens.

In an embodiment, the invention includes pharmaceutical formulations comprising pre-mix compositions comprising conjugated estrogens.

In another embodiment, the invention includes processes for preparing pharmaceutical formulations comprising conjugated estrogens.

In an embodiment, the invention includes processes for preparing pharmaceutical formulations comprising conjugated estrogens or pre-mixes, wherein conjugated estrogens or pre-mix compositions are incorporated into the formulation through a binder solution or are added extra granularly or by serial/geometric dilution.

In an embodiment, the invention includes pre-mix compositions or pharmaceutical formulations comprising conjugated estrogens or pre-mixes, wherein relative standard deviation value for conjugated estrogens concentration is not more than about 6.

In an aspect, the present invention includes stable pre-mix compositions comprising conjugated estrogens.

In an embodiment, the invention includes stable pre-mix compositions wherein moisture content is not more than about 2% w/w of total composition.

In an embodiment, the invention includes stable pharmaceutical formulations comprising conjugated estrogens.

In an embodiment, the invention includes stable pharmaceutical formulations comprising pre-mix compositions of conjugated estrogens wherein moisture contents not more than about 6% w/w of total composition.

An aspect of the present invention relates to particle size distributions of conjugated estrogens, wherein D₁₀ is in a range of about 1 to 20 μm, D₅₀ is in a range of about 10 to about 50 μm, D₉₀ is in a range of about 40 to about 150 μm, and D_{[4,3]} is in a range of about 15 to about 75 μm.

In an embodiment the invention includes particle size distribution of pre-mix compositions, wherein D₁₀ is in a range of about 0.1 to about 20 μm or about 0.1 to about 10 μm, D₅₀ is in a range of about 1 to about 75 μm or about 5 to about 50 μm, and D₉₀ is in a range of about 20 to about 150 μm or about 25 to about 100 μm.

In an embodiment, the invention includes bulk densities and tapped densities of pre-mix compositions comprising conjugated estrogens, wherein a bulk density is in the range of about 0.2 to about 0.6 g/ml or 0.2 to about 0.45 g/ml, and a tapped density is in the range of about 0.2 to about 0.6 g/ml or 0.3 to about 0.5 g/ml.

In an embodiment the invention includes pharmaceutical formulations comprising conjugated estrogens, wherein conjugated estrogens are released according to the following dissolution profile when tested in USP Apparatus II, with 900 ml of purified water (degassed) and 50 rpm stirring:

a) Less than about 35% of conjugated estrogens is released within about one hour.

b) Less than about 65% of conjugated estrogens is released within about 2 hours.

c) About 30% to about 100% of conjugated estrogens is released within about 5 hours.

d) Not less than about 60% of conjugated estrogens is released within about 8 hours.

In embodiments the invention includes pharmaceutical formulations comprising conjugated estrogens, wherein conjugated estrogens are released according to the following dissolution profile when tested in USP Apparatus II, with 900 ml of pH 4.5 acetate buffer and 50 rpm stirring:

a) About 2% to about 30% of conjugated estrogens is released within about one hour.

b) About 5% to about 55% of conjugated estrogens is released within about 2 hours.

c) About 60% to about 100% of conjugated estrogens is released within about 5 hours.

d) Not less than about 70% of conjugated estrogens is released within about 8 hours.

In an aspect, the invention includes methods of using pharmaceutical compositions of the present invention.

### BRIEF DESCRIPTION OF THE DRAWING

FIG. 1 is a schematic diagram of spray drying apparatus used for preparing pre-mix compositions comprising conjugated estrogens, in Example 1.

### DETAILED DESCRIPTION

An aspect of the present invention relates to pre-mix compositions comprising conjugated estrogens and at least one pharmaceutically acceptable carrier, and processes for the preparation of pre-mix compositions.

Aspects of the invention relate to pharmaceutical formulations comprising pre-mix compositions comprising conjugated estrogens and processes for preparing them. The invention also includes therapeutic uses and methods of treatment employing pre-mix compositions comprising conjugated estrogens and their pharmaceutical formulations.

Estrogens are used in medicine for hormone replacement therapy. In particular, estrogen mixtures are used for the treatment and prophylaxis of the disorders of the climacteric period, which occur in women after natural or artificial menopause. In these case, natural mixtures of conjugated estrogens such as are found in the urine of pregnant mares have proved particularly effective and readily compatible.

The natural mixtures of estrogens contained in pregnant mare urine (PMU) are largely present in conjugated form, i.e., as sulfuric acid semi-ester sodium salts. The natural conjugated estrogens obtained from PMU contains at least 10 estrogen compounds that are sulfate esters of the ring B saturated estrogens: estrone, 17β-estradiol, 17α-estradiol, and the ring B unsaturated estrogens: equilin, 17β-dihydroequilin, 17α- dihydroequilin, equilenin, 17β-dihydroequilenin, 17α-dihydroequilenin, and delta-8-estrone. Bioassays and estrogen receptor binding studies indicate that all 10 estrogens are biologically active, and the present invention may utilize such mixtures, or include only selected or individual estrogenic components. These conjugated estrogens may be of synthetic or natural origin.

Conjugated estrogens are a potent drug with administered doses such as 0.3 mg, 0.45 mg, 0.625 mg, 0.9 mg, or 1.25 mg. It is a mixture having two main constituents, estrone and equilin, and several other concomitant constituents. To maintain the levels of these constituents uniformly in a pharmaceutical formulation and to develop a stable formulation is a difficult task.

It is sometimes necessary or desired to administer high potency drugs in solid oral dosage forms containing very small, but pharmacologically effective amount of the drug. Many approaches are used to improve the content uniformity for low dose drugs including micronized particles of drug so that there is uniform distribution of drug in the blend, distributing through binder solution during granulation process, coating the drug solution onto inert particles etc. But many of such high potent drugs cannot be formed by conventional methods into very small particles of highly uniform size and may affect the stability of drug. As a result it is difficult to provide solid unit dosage forms containing such drugs that will pass the USP content uniformity test as set forth in United States Pharmacopoeia.

It has now been discovered that when a solution of conjugated estrogens, optionally with at least one carrier, is spray dried, there is formed a stable, homogenously distributed free-flowing powder, which exhibits good processing characteristics, and when these granules are processed together with certain pharmaceutically acceptable excipients in the presence of a solvent there is formed formulations of conjugated estrogens with good content uniformity and stability.

The term “conjugated estrogens” for purposes of the present invention includes crystalline or amorphous forms, or salts or solvates or co-crystals.

The term “pre-mix compositions” for purposes of the present invention includes the compositions comprising conjugated estrogens and at least one pharmaceutical carrier in intimate contact.

The term “intimate contact” for purposes of the present invention is defined as a combination of conjugated estrogens and a pharmaceutical acceptable carrier, in a form where the individual components cannot be distinguished, using techniques such as optical microscopy. The compositions can be formed by processes such as, but not limited to, coprecipitation from a solvent.

The term “pharmaceutical formulations” for purposes of the present invention includes solid oral dosage forms such as tablets, capsules, granules, pills, sachets etc comprising pre-mix compositions of conjugated estrogens and at least one pharmaceutical acceptable excipient.

Alternatively, the pharmaceutical formulations may be in monolithic forms or in multi-particulate systems.

Conjugated estrogens are a highly water-soluble drug. But in spite of its high solubility and because of the low dosage form content; formulations comprising conjugated estrogens have a problem with content uniformity. The distribution of the drug substance in the blend or content uniformity of drug in the blend with excipients is important in order to obtain formulations with uniform drug content. Uniformity of content may be achieved to some extent by reduction in particle sizes, but the reduction of particle size involves cumbersome processing and during the processing the stability of drug may be affected.

Content uniformity of conjugated estrogens may be improved by forming a pre-mix composition of conjugated estrogens, wherein conjugated estrogens are in intimate contact with at least one pharmaceutical excipient. By using the pre-mix compositions the quantity of the drug-containing component to be incorporated into the formulation will be increased, and larger quantities are easier to handle and blend uniformly.

In an embodiment, the present invention includes pre-mix compositions of conjugated estrogens and at least one pharmaceutical excipient.

In an embodiment the invention includes processes to prepare the pre-mix compositions of conjugated estrogens and at least one pharmaceutical carrier.

It has been observed that by spray drying a solution or dispersion of conjugated estrogens and at least one pharmaceutical carrier, there is formed a dry solid having desired parameters that is advantageously suitable for producing solid dosage formulations such as tablets and capsules.

Spray drying provides transformation of feed material from a fluid state into dried particulate form, by spraying the feed into a hot drying medium. It is a continuous particle-processing drying operation. The feed can be a solution, suspension, dispersion, emulsion or slip. The dried product can be in the form of powders, granules, or agglomerates depending upon the physical and chemical properties of the feed, the dryer design and final powder properties desired. Feed material is finely atomized and is introduced in the drying chamber along with heated air. The mixture of atomized feed and hot air moves towards the air exhaust of drying chamber. The time taken by this mixture to travel up to air exhaust is called a residence time of drying. During this residence time, the feed droplets lose their moisture to the hot air and are converted into dry powder particles. Heated air absorbs this moisture, so its absolute humidity increases while its temperature is reduced. The feed droplets, while losing moisture to heated air, remain at temperatures much below the air temperature and are exposed to heated air for a very short time. Hence, spray drying is essentially known as “low-temperature drying.” The temperature of a feed droplet is the wet bulb temperature at the prevailing relative humidity.

The dry powder falling on to the conical portion of a drying chamber slides down to the bottom of the drying chamber, with the assistance of vibration, and is collected in a collection bottle. The air leaving the drying chamber entrains some of the dry powder, which is recovered in the cyclone separators 1 and 2, and collected through a rotary airlock. The air leaving the cyclone separators still has traces of dry powder, which is further recovered by scrubbing in a scrubber. In the scrubber, the dry powder particles are retained and the exiting air is dust-free and clean, and can be exhausted to the atmosphere. Removal of air from the scrubber is assisted by vacuum from an aspirator assembly.

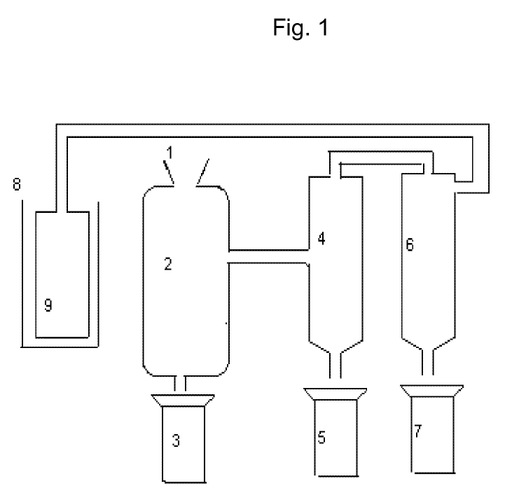

In the spray drying apparatus of FIG. 1, 1 represents a spray inlet port, 2 represents a drying chamber, 3 represents a first collector, 4 represents a first cyclone, 5 represents a second collector, 6 represents a second cyclone, 7 represents a third collector, 8 represents a vacuum source, and 9 represents a scrubber.

The desired parameters of the pre-mix compositions include but are not limited to flowability, particle size distribution (determined by sieve analyzer or a laser diffraction particle size analyzer, such as is sold by Malvern Instruments Ltd., Malvern, Worcestershire, United Kingdom), moisture content (such as determined by Karl Fischer (KF) apparatus or infrared moisture balance), bulk density, tapped density, compressibility index, Hausner ratio (determined by USP density apparatus), content uniformity, span value, etc.

Bulk density is a property of particulate materials. It is the mass of many particles of the material divided by the volume they occupy. The volume includes the space between particles as well as the space inside the pores of individual particles. Bulk density is not an intrinsic property of a material; it can change depending on how the material is handled. For example, particles poured into a cylinder will have a particular bulk density. If the cylinder is disturbed, the particles will move and settle closer together, resulting in a higher bulk density. For this reason, the bulk density of powders is usually reported both as “freely settled” and “tapped” densities (where the tapped density refers to the bulk density of the powder after a specified compaction process, usually involving vibration of the container).

In an aspect, the invention includes spray-drying processes for preparing pre-mix compositions comprising conjugated estrogens, wherein an embodiment of a process comprises:

1) Dissolving or dispersing drug in a suitable solvent.

2) Dissolving or dispersing a suitable carrier in the solution or dispersion.

3) Spray drying the solution or dispersion from step 2) to evaporate solvent.

4) Optionally, drying the solid obtained from step 3).

Suitable solvents that can be used for to prepare pre-mix compositions of conjugated estrogens include, but are not limited to: alcohols such as methanol, ethanol, isopropyl alcohol, n-propanol, and the like; halogenated hydrocarbons such as dichloromethane, 1,2-dichloroethane, chloroform, carbon tetrachloride, and the like; ketones such as acetone, ethyl methyl ketone, methyl isobutyl ketone, and the like; esters such as ethyl acetate, n-propyl acetate, n-butyl acetate, t-butyl acetate, and the like; ethers such as diethyl ether, dimethyl ether, diisopropyl ether, 1,4-dioxane, and the like; hydrocarbons such as toluene, xylene, n-heptane, cyclohexane, n-hexane, and the like; nitriles such as acetonitrile, propionitrile, and the like; mixtures of any two or more thereof; and their combinations with water.

Suitable pharmaceutical carriers that are useful in preparing the pre-mix compositions include but are not limited to lactose, mannitol, sorbitol, dicalcium phosphate, tribasic calcium phosphate, microcrystalline cellulose, hydroxyalkyl celluloses such as hydroxymethylcelluloses, hydroxyethylcelluloses, hydroxypropyl celluloses, hydroxyethyl methylcelluloses, hydroxypropyl methylcelluloses, polyvinylpyrrolidones, and the like. In embodiments a carrier will comprise a sugar or sugar alcohol, a hydroxyalkylcellulose such as a hydroxypropylcellulose and/or hydroxypropyl methylcellulose, an alkyl cellulose such as a methylcellulose or ethyl cellulose, calcium carbonate, magnesium carbonate, calcium sulphate, and/or any combinations thereof.

In an embodiment, the present invention includes pre-mix compositions wherein weight ratios of conjugated estrogens to pharmaceutical carrier are in the range of about 1:1 to about 1:75.

In an embodiment, the present invention includes pre-mix compositions formed from weight ratios of conjugated estrogens to solvent in the range of about 1:50 to about 1:500.

In another embodiment the present invention includes pre-mix compositions wherein the concentration of conjugated estrogens is at least about 0.1 percent by weight of the total composition.

Due to the increased awareness of bioavailability and safety, compendia authorities have implemented a multi-stage content uniformity test for low dose drugs, which includes 1) assaying ten dosage foerms to ensure that the relative standard deviation (RSD) of active ingredient content is less than or equal to 6% and no value is outside 85-115%; and 2) assaying twenty more tablets to ensure that the RSD for all thirty dosage forms is less than or equal to 7.8%, not more than one value is outside 85-115% and no value is outside 75-125% of stated content.

s=[Σ(Xᵢ−X⁻)²÷n−1]^{1/2}

RSD=100s÷X⁻

In the above formulae, s is the standard deviation; RSD is the relative standard deviation; Xᵢ includes X₁, X₂, X₃ . . . Xₙ, which are individual amounts of the tested samples expressed as percentages of the labeled amount of drug substance in each sample; X⁻ is the mean of the values obtained from the samples tested, expressed as a percentage of the labeled amount of drug substance in each sample; and n is the number of units tested.

Content uniformity of the dosage forms can be determined by performing an HPLC assay to measure the amount of drug in each unit dosage form, and comparing the amount of drug in each dosage form.

In an embodiment the invention includes pre-mix compositions of conjugated estrogens wherein a relative standard deviation of conjugated estrogen content is less than about 6.

Uniform particle size distributions of conjugated estrogens and also the pre-mix compositions are desired to get content uniformity in the formulation.

Particle sizes for a powdered material can generally be given in terms of parameters such as D₁₀, D₅₀, D₉₀, and D_{[4,3]} that are used routinely to describe the particle distribution. Values are expressed as volume or weight or surface percentages. Dₓ as used herein is defined as the size of particles where x volume or weight percent of the particles have sizes less than the value given. D_{[4,3]} for example is the volume mean diameter of the conjugated estrogens or other powder compositions. D₉₀ for example means that 90% of the particles are below a given particle size. Particle size or particle size distribution of the pre-mix compositions of present invention are determined using techniques that are known to the person skilled in the art including but not limited to sieve analysis, size analysis by laser diffraction such as a Malvern particle size analyzer (Malvern Instruments Ltd., Malvern, Worcestershire, United Kingdom) and the like. Pre-mix compositions of conjugated estrogens of the present invention are fine, uniform and agglomerate free. The desired particle size distribution may be obtained by techniques such as sieving or air jet milling and can conveniently be measured by a laser light scattering method.

In an embodiment, the present invention relates to particle size distributions of conjugated estrogens, wherein D₁₀ is in the range of about 1 to 20 μm, D₅₀ is in the range of about 10 to about 50 μm, D₉₀ is in the range of about 40 to about 150 μm, and D_{[4,3]} is in the range of about 15 to about 75 μm.

In an embodiment, the invention includes pre-mix compositions of conjugated estrogens wherein the particle size distribution has D₁₀ is in a range of about 0.1 to about 20 μm or about 0.1 to about 10 μm, D₅₀ is in a range of about 1 to about 75 μm or about 5 to about 50 μm, and D₉₀ is in a range of about 20 to about 150 μm or about 25 to about 100 μm.

In an embodiment, the invention includes bulk densities and tapped densities of pre-mix compositions comprising conjugated estrogens, wherein a bulk density is in the range of about 0.2 to about 0.6 g/ml or 0.2 to about 0.45 g/ml, and a tapped density is in the range of about 0.2 to about 0.6 g/ml or 0.3 to about 0.5 g/ml.

It has been observed that presence of moisture contents greater than acceptable levels would cause significant degradation of the conjugated estrogens over a short period of time. In an embodiment, a conjugated estrogens pre-mix will have a moisture content not exceeding about 4 percent by weight.

Yet another embodiment of the invention includes stable pre-mix compositions of conjugated estrogens.

In a further embodiment the invention includes stable pre-mix compositions of conjugated estrogens, wherein a loss on drying is not more than about 5% by weight.

The present invention further relates to pharmaceutical formulations comprising conjugated estrogens.

Further, the present invention relates to pharmaceutical formulations comprising pre-mix compositions of conjugated estrogens.

The formulations of the present invention may be any dosage form such as tablets, capsules, pills, granules, sachets, gels, creams, solutions, etc.

In an embodiment the invention includes solid oral dosage forms comprising conjugated estrogens.

The dose included in the pre-mix compositions or its formulations may be any dose required to achieve a specific therapeutic effect, and may vary depending on the specific treatment indicated, and on the specific conjugated estrogen included in the tablet. However, in general, administered doses of conjugated estrogens included in tablets can range from about 0.2 mg to about 3 mg, per dosage unit.

The pharmaceutical formulations may further comprise pharmaceutical excipients which include but are not limited to any one or more of diluents, disintegrants, binders, glidants, lubricants, solvents, stabilizers, and colouring agents.

### Diluents:

Various useful diluents include but are not limited to starches, lactose, mannitol, cellulose derivatives and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline celluloses and powdered cellulose. Examples of crystalline cellulose products include but are not limited to CEOLUS™ KG801, Avicel™ PH 101, PH102, PH301, PH302 and PH-F20, microcrystalline cellulose 114, and microcrystalline cellulose 112. Other useful diluents include but are not limited to carmellose, sugar alcohols such as sorbitol and xylitol, calcium carbonate, magnesium carbonate, dibasic calcium phosphate, and tribasic calcium phosphate.

### Disintegrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (FMC-Asahi Chemical Industry Co., Ltd.), crospovidones, examples of commercially available crospovidone products including but not being limited to crosslinked povidone, Kollidon™ CL [manufactured by BASF (Germany)], Polyplasdone™ XL, XI-10, and INF-10 [manufactured by ISP Inc. (USA)], and low-substituted hydroxypropylcelluloses. Examples of low-substituted hydroxypropylcellulose include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide, and starches.

### Binders:

Various useful binders include but are not limited to hydroxypropylcelluloses (Klucel™ LF), hydroxypropyl methylcelluloses (Methocel™), polyvinylpyrrolidones or povidones (PVP-K25, PVP-K29, PVP-K30), powdered acacia, gelatin, guar gum, carbomers (Carbopol™), methylcelluloses, polymethacrylates, and starches.

### Glidants:

Various useful glidants or antisticking agents include but are not limited to talc, silica derivatives, and colloidal silicon dioxide.

### Solvents:

Various solvents that are useful in formulation processing include, but are not limited to, water, lower alcohols like methanol, ethanol, and isopropanol, acidified ethanol, acetone, polyols, polyethers, oils, esters, alkyl ketones, methylene chloride, castor oil, ethylene glycol monoethyl ether, diethylene glycol monobutyl ether, diethylene glycol monoethyl ether, dimethylsulphoxide, N,N-dimethylformamide, and tetrahydrofuran.

### Stabilizers:

Various stabilizers that are useful include but not limited to: alkali metal salts such as sodium carbonate, sodium bicarbonate etc; alkaline earth metal salts such as magnesium carbonate, calcium carbonate, tricalcium phosphate, dibasic calcium phosphate, etc.; meglumine; alkali metal salts of organic acids, such as disodium tartrate, sodium citrate, etc.; and the like.

### Colorants:

Various useful colorants include but are not limited to Food Yellow No. 5, Food Red No. 2, Food Blue No. 2, and the like, food lake colorants, and iron oxides.

### Film-Forming Agents:

Various film-forming agents that can be used include but are not limited to cellulose derivatives such as soluble alkyl- or hydroalkyl-cellulose derivatives such as methyl celluloses, hydroxymethyl celluloses, hydroxyethyl celluloses, hydroxypropyl celluloses, hydroxymethyethyl celluloses, hydroxypropyl methylcelluloses, sodium carboxymethyl celluloses, etc., acidic cellulose derivatives such as cellulose acetate phthalates, cellulose acetate trimellitates and methylhydroxypropylcellulose phthalates, polyvinyl acetate phthalates, etc., insoluble cellulose derivatives such as ethylcelluloses and the like, dextrins, starches and starch derivatives, polymers based on carbohydrates and derivatives thereof, natural gums such as gum Arabic, xanthans, alginates, polyacrylic acid, polyvinylalcohols, polyvinyl acetates, polyvinylpyrrolidones, polymethacrylates and derivatives thereof (Eudragit™), chitosan and derivatives thereof, shellac and derivatives thereof, waxes, and fat substances.

The coatings may be applied using methods such as film coating, press coating, tablet coating, encapsulating or microencapsulating.

If required, the films may contain additional adjuvants for coating processing such as plasticizers, polishing agents, colorants, pigments, antifoam agents, opacifiers, antisticking agents, and the like.

### Plasticizers:

Representative plasticizers include but are not limited to castor oil, diacetylated monoglycerides, dibutyl sebacate, diethyl phthalate, glycerin, polyethylene glycols, propylene glycols, triacetin, and triethyl citrate. Also mixtures of plasticizers may be utilized. The type of plasticizer depends upon the type of coating agent. A plasticizer is frequently present in an amount ranging from 5% (w/w) to 30% (w/w), based on the total weight of the film coating.

An opacifier like titianium dioxide may also be present in an amount ranging from about 10% (w/w) to about 20% (w/w), based on the total weight of the coating. When coloured tablets are desired then the colour is frequently applied in the coating. Consequently, colouring agents and pigments may be present in the film coating. Various colouring agents include but are not limited to iron oxides, which can be red, yellow, black or blends thereof.

Anti-adhesives are frequently used in film coating processes to avoid sticking effects during film formation and drying. An example of an anti-adhesive for this purpose is talc.

Suitable polishing agents include polyethylene glycols of various molecular weights or mixtures thereof, talc, surfactants (e.g. glycerol mono-stearate and poloxamers), fatty alcohols (e.g., stearyl alcohol, cetyl alcohol, lauryl alcohol and myristyl alcohol) and waxes (e.g., carnauba wax, candelilla wax and white wax). In an embodiment, polyethylene glycols having molecular weights of 3,000-20,000 are employed.

In addition to above the coating ingredients, sometimes pre-mixed coating products such as those sold by Colorcon as Opadry™ will be used. The products require only mixing with a liquid before use.

### Processes for Preparing Formulations:

The pharmaceutical formulations may be prepared using any one or more of different formulation techniques such as physical mixing, blending, wet granulation, dry granulation, direct compression, fluid bed granulation, etc.

An aspect of the present invention is further directed to processes for preparing pharmaceutical formulations comprising conjugated estrogens or pre-mix compositions thereof, wherein an embodiment of a process comprises:

a) Sifting the drug or its pre-mixes, diluents, disintegrants and other excipients through a sieve.

b) Dry mixing sifted drug, diluents, and disintegrants and other excipients.

c) Granulating the dry mix with a binder solution.

d) Drying the granules.

e) Passing the dried granules through a sieve.

f) Mixing the dried granules with sifted extragranular materials and blending.

g) Compressing the blend into tablets or filling into capsules.

h) Coating tablets with a coating dispersion.

Optionally, step b) materials may be blended with extragranular excipients and compressed into tablets or may be filled into capsules.

Optionally, step b) materials may be compressed to form slugs which are further milled through a sieve and blended with extragranular excipients and compressed into tablets or filled into capsules.

Alternatively, from step a) drug is omitted and the excipients are mixed and granulated with a drug solution.

Alternatively, from step a) drug is omitted and the excipients are granulated with a solvent and dried. Then subject the placebo granules and drug to serial/geometric dilution.

In embodiments, pharmaceutical formulations comprising a conjugated estrogen pre-mix have moisture contents not exceeding about 6 percent by weight.

In an embodiment of the invention, pharmaceutical formulations comprising conjugated estrogens can also include other drugs used in estrogen replacement therapy. Specific progestins that may be used include without limitation: progesterone, medroxyprogesterone, and a variety of synthetic progestins and their salts, esters, and derivatives that are generally known and used in the oral contraceptive area. Specific androgens that may be used include without limitation, testosterone, methyltestosterone, and other known derivatives and their esters and salts, including deconoate, cypionate, propionate, etc. Any of these hormones can also be micronized.

In an embodiment, the invention includes the use of packaging materials such as containers and lids of high-density polyethylene (HDPE), low-density polyethylene (LDPE) and or polypropylene and/or glass, and blisters or strips composed of aluminum or high-density polypropylene, polyvinyl chloride, polyvinylidene dichloride, and combinations thereof.

The dosage forms can be subjected to in vitro dissolution evaluation according to Test 711 “Dissolution” in United States Pharmacopoeia 29, United States Pharmacopoeial Convention, Inc., Rockville, Md., 2005, to determine the rate at which conjugated estrogens are released from the dosage forms, and conjugated estrogens can conveniently be determined in solutions using high performance liquid chromatography.

The pharmaceutical dosage forms of the present invention are intended for oral administration to a patient in need thereof.

In determining bioequivalence between two products, such as a commercially-available product and a test product, pharmacokinetic studies can be conducted whereby the products are administered in a cross-over study to volunteer subjects. Serum plasma samples are obtained at regular intervals following dosing and assayed for parent drug (or sometimes metabolite) concentrations. For a pharmacokinetic comparison, the plasma concentration data are used to assess key pharmacokinetic parameters including area under the plasma concentration-time curve (AUC), peak plasma drug concentration (Cₘₐₓ) and time to peak plasma concentration (Tₘₐₓ).

In an embodiment the invention includes pharmaceutical formulation containing 0.625 mg of conjugated estrogen and producing: estrone Cₘₐₓ values about 58 pg/mL to about 90 pg/mL, AUC₀₋ₜ values about 2340 pg·hour/mL to about 3658 pg·hour/mL, and AUC₀₋ₘ values about 5754 pg·hour/mL to about 8991 pg·hour/mL; and equilin Cₘₐₓ values about 10 pg/mL to about 16 pg/mL, AUC₀₋ₜ values about 187 pg·hour/mL to about 292 pg hour/mL, and AUC_{0-∞} values about 242 pg·hour/mL to about 378 pg·hour/mL; in plasma after oral administration of a single dose to healthy humans under fasting conditions.

In another embodiment the invention includes pharmaceutical formulations containing 0.625 mg of conjugated estrogen and producing: estrone Cₘₐₓ values about 57 pg/mL to about 89 pg/mL, AUC₀₋ₜ values about 1903 pg·hour/mL to about 2974 pg·hour/mL, and AUC_{0-∞} values about 2989 pg·hour/mL to about 4670 pg·hour/mL; and equilin Cₘₐₓ values about 11 pg/mL to about 18 pg/mL, AUC₀₋ₜ values about 174 pg·hour/mL to about 272 pg·hour/mL, and AUC_{0-∞} values about 216 pg·hour/mL to about 338 pg·hour/mL; in plasma after oral administration of a single dose to healthy humans under fed conditions.

Further embodiments include pharmaceutical formulations providing Cₘₐₓ and AUC₀₋ₜ values that do not vary by more than about 20% between fed state administration and fasted state administration to healthy humans, i.e., there is no significant food effect observed.

Certain specific aspects and embodiments of the invention will be further described in the following examples, which are provided only for purposes of illustration and are not intended to limit the scope of the invention in any manner.

### EXAMPLE 1

### Pre-Mix Compositions Comprising Conjugated Estrogens

Quantity (parts by weight)

| Ingredient | 1A | 1B |
|---|---|---|
| Conjugated estrogens | 1 | 1 |
| Lactose monohydrate | 10 | 10 |
| Tribasic calcium phosphate | 10 | — |
| Water* | — | 126.5 |
| Methanol* | 210 | — |

*Evaporates during processing.

Particle size distribution values of conjugated estrogens used in this example are shown below:

| | |
|---|---|
| D₁₀ | 78.4 μm |
| D₅₀ | 463.5 μm |
| D₉₀ | 833.3 μm |

Manufacturing Process:

1) Conjugated estrogens were dissolved in methanol or water.

2) Lactose monohydrate and tricalcium phosphate (for Example 1A) were dispersed or dissolved into the step 1) solution.

3) The dispersion or solution was spray dried in a Labultima Model No. LU 228 Advanced spray dryer according to FIG. 1 with the conditions: inlet temperature 50° C. for 1A, and 120 to 135° C. for 1B; outlet temperature: about 48° C.; feed rate: 4 mL/minute for 1A, and 2.5 to 3 mL/minute for 1B; atomization pressure: about 0.6 bar for 1A, and 1.8 bar for 1B.

The particle size distribution, loss on drying at 105° C. (LOD), and bulk density data for the pre-mix compositions are shown in Table 1.

TABLE 1

| | | | | | | Bulk Density |
|---|---|---|---|---|---|---|
| Material | Sample | D₁₀ | D₅₀ | D₉₀ | LOD | (g/mL) |
| 1A | Pre-mix | 2.42 | 18.105 | 43.713 | 1.27 | 0.312 |
| | collected in | | | | | |
| | cyclones | | | | | |
| | Pre-mix | 2.787 | 21.681 | 51.224 | 1.3 | 0.296 |
| | collected in | | | | | |
| | first collector | | | | | |

| | | | | | |
|---|---|---|---|---|---|
| 1B | 1.32 | 15.26 | 33.65 | 2.78 | 0.38 |

Pharmaceutical formulation containing a pre-mix of 1B.

| Ingredient | mg/Tablet |
|---|---|
| Conjugated estrogens pre-mix | 3.582 |
| from 1B* | |
| Lactose monohydrate | 120 |
| (Pharmatose DCL 11) | |
| Tricalcium phosphate | 12 |
| Methocel K100M DC | 21 |
| Silicon dioxide (Syloid 244 FP) | 1.2 |
| Talc | 2.4 |
| Magnesium stearate | 2.4 |

*3.582 mg of conjugated estrogens active ingredient provides 0.252 mg of estrone and equilin.

Manufacturing Process:

1) Lactose monohydrate is sifted through an ASTM #100 mesh sieve to separate fine particles. The retained particles are sifted through an ASTM #40 mesh sieve and particles not passing through the sieve are discarded.

2) Conjugated estrogens pre-mix and ASTM 40# mesh sieve sifted tricalcium phosphate and an equivalent quantity of fines of ASTM #100 mesh sieve of lactose monohydrate from step 1) are sifted through ASTM #100 mesh sieve and blended for 5 minutes.

3) The blend of step 2) is mixed with an equal quantity of fines of ASTM #100 mesh sieve fraction lactose monohydrate particles from step 1) and blended for about 5 minutes.

4) Methocel K100M CR, Syloid 244 FP and talc are sifted through an ASTM #40 mesh sieve.

5) Steps 3) and 4) ingredients and remaining quantity of lactose monohydrate from step 1) are blended together for about 30 minutes.

6) Magnesium stearate is sifted through an ASTM #60 mesh sieve, is added to step 5), and blended for about 5 minutes.

7) The lubricated blend is compressed into tablets.

The tablets are analyzed for content uniformity, and an average content of (estrone+equilin) is 85.4% of the label total estrogens, with a relative standard deviation about 2.

### EXAMPLES 2-4

### Conjugated Estrogens Tablets

| | mg/Tablet | | |
|---|---|---|---|
| Ingredient | Example 2 | Example 3 | Example 4 |
| Conjugated estrogens‡ | 1.9 | 1.9 | 3.61 |
| Lactose monohydrate | 87 | 98.6 | 173.99 |
| Hydroxypropyl methylcellulose | 18 | 18 | 36 |
| (Methocel ™ K 100 M CR) | | | |
| Isopropyl alcohol* | 75.5 | 83.95 | 151.2 |
| Water* | 50.4 | 55.97 | 100.8 |
| Light magnesium carbonate | — | 0.3 | — |
| Tribasic calcium phosphate | 12 | — | 24 |
| Magnesium stearate | 1.2 | 1.2 | 2.4 |
| Ethylcellulose 7 cps | 4.86 | 4.86 | 20.57 |
| Hydroxypropyl methylcellulose | 4.86 | 4.86 | 20.57 |
| (Methocel E 5 Premium) | | | |
| Polyethylene glycol 400 (Lutrol ™ | 0.49 | 0.49 | 2.06 |
| E 400) | | | |
| Isopropyl alcohol* | 87.3 | 87.3 | 369.36 |
| Methylene chloride* | 87.3 | 87.3 | 369.36 |
| Water* | 19.4 | 19.4 | 82.08 |
| Opadry ™ Brown** | 2.6 | 2.6 | — |
| Opadry Yellow*** | — | — | 5.66 |
| Isopropyl alcohol* | 24.7 | 24.7 | 53.77 |
| Methylene chloride* | 24.7 | 24.7 | 53.77 |

*Evaporates during processing.
‡1.9 mg of conjugated estrogens active ingredient provides 0.525 mg of estrone and equiline. ‡3.8 mg of conjugated estrogens active ingredient provides 1.05 mg of estrone and equiline.
**Opadry Brown is a preformulated coating product that contains hypromellose, iron oxide red, polyethylene glycol, titanium dioxide, and FD&C blue #2 aluminum lake, and is supplied by Colorcon.

Manufacturing Process:

1) Lactose monohydrate and Methocel K 100 M CR were sifted through an ASTM #40 mesh sieve.

2) Step 1) components were loaded into a fluidized bed coater and mixed for about 5 minutes.

3) The mixture of isopropyl alcohol (first quantity) and water (first quantity) was sprayed on the step 2) material to form granules.

4) Granules obtained from step 3) were dried and sifted through an ASTM #60 mesh sieve.

5) Step 4) granules were sifted through an ASTM #100 mesh sieve to prepare a fine particle fraction and a coarse particle fraction.

6) Conjugated estrogens and fine particles of step 5) were sifted through an ASTM #100 mesh sieve.

7) The mass of step 6), Tribasic calcium phosphate or light MgCO₃ and magnesium stearate were co-sifted through an ASTM #100 mesh sieve in geometric proportion.

8) Equivalent amount of placebo granules from step 5) and the mixture from step 7) were co-sifted through an ASTM #60 mesh sieve.

9) Remaining quantity of placebo granules and the mass at step 8 were co-sifted through ASTM #60 for couple of times and blended for 30 minutes.

10) Step 9) blend was compressed into tablets.

11) Methocel E 5 cps, ethylcellulose 7 cps, and polyethylene glycol 400 were dissolved in isopropyl alcohol (second quantity), methylene chloride and water (second quantity) to form an extended-release coating composition.

12) The compressed tablets of step 10) were coated with the coating composition of step 11).

13) The tablets of step 12) were film coated using an Opadry dispersion in isopropyl alcohol (third quantity) and methylene chloride (second quantity).

The tablets were subjected to in vitro dissolution testing in USP apparatus type 11 with 900 mL of purified water (degassed), temperature 37±0.5° C., and 50 rpm rotation, and compared with PREMARIN® 0.625 mg and 1.25 mg “Reference” tablets. The results are shown in Table 2.

TABLE 2

| | Cumulative % of Drug Dissolved | | | | |
|---|---|---|---|---|---|
| | 0.625 mg Tablets | | | 1.25 mg Tablets | |
| Hours | Example 2 | Example 3 | Reference | Example 4 | Reference |
| 1 | 27 | 31 | 18 | 9 | 7 |
| 2 | 47 | 51 | 44 | 27 | 27 |
| 5 | 86 | 88 | 88 | 67 | 73 |
| 8 | 102 | 100 | 99 | 90 | 94 |
| 10 | 104 | 102 | 100 | 101 | 100 |

Tablets prepared in Example 2 and Example 3, and PREMARIN® 0.625 mg (Reference), were stored at 40° C. and 75% relative humidity (“RH”) in closed HDPE bottles, containing a molecular sieve desiccant. The estrone and equiline contents, and weight ratios of equiline to estrone, of the conjugated estrogens tablets, were determined at intervals and the data are shown in Table 3. Values for estrone, equilin, and their total, are expressed as percentages of the conjugated estrogen content.

TABLE 3

| | Example 2 | | Example 3 | | | PREMARIN ® | |
|---|---|---|---|---|---|---|---|
| Component | Initial | 3 Mo. | Initial | 3 Mo. | 6 Mo. | Initial | 3 Mo. |
| Estrone | 56.17 | 56.81 | 67.28 | 67.88 | 70.76 | 48.70 | 53.45 |
| Equiline | 29.21 | 27.81 | 28.25 | 30.86 | 28.45 | 26.04 | 25.27 |
| Total | 85.39 | 84.6 | 95.53 | 98.73 | 99.21 | 74.74 | 78.7 |
| Equiline: | 0.52 | 0.49 | 0.42 | 0.45 | 0.4 | 0.54 | 0.47 |
| Estrone | | | | | | | |

A pharmacokinetic study was conducted. Pharmacokinetic parameters Cₘₐₓ (maximum concentration of drug in the plasma), AUC₀₋ₜ (area under the curve from the time of administration to the last time of a measurable plasma concentration), and AUC_{0-∝} (area under the curve from the time of administration to time infinity) were determined for tablets of Example 2 (test product, or “T”) and PREMARIN® 0.625 mg tablets (reference product, or “R”), in a crossover study involving 14 subjects, with drug administered both in fasting and fed conditions. The average results for estrone and equilin are shown in Table 4.

TABLE 4

| | Fasting | | | Fed | | |
|---|---|---|---|---|---|---|
| | Cₘₐₓ | | AUC_{0-∝} | Cₘₐₓ | | |
| | (T/R, | AUC₀₋ₜ | (T/R, | (T/R, | AUC₀₋ₜ | AUC_{0-∝} |
| Component | %) | (T/R, %) | %) | %) | (T/R, %) | (T/R, %) |
| Estrone | 90.33 | 89.57 | 101.28 | 88.01 | 89.44 | 87.63 |
| Equilin | 89.34 | 91.49 | 98.72 | 99.21 | 111.46 | 107.54 |

Average values from the above study are further shown below, where Cₘₐₓ values are in pg/mL, and AUC values are in pg·hour/mL.

| | | Fasting | | | Fed | | |
|---|---|---|---|---|---|---|---|
| Component | | Cₘₐₓ | AUC₀₋ₜ | AUC_{0-∝} | Cₘₐₓ | AUC₀₋ₜ | AUC_{0-∝} |
| Test | Estrone | 72 | 2926 | 7193 | 71 | 2379 | 3736 |
| | Equilin | 13 | 234 | 302 | 14 | 218 | 270 |
| Refer- | Estrone | 82 | 3108 | 5733 | 80 | 2756 | 5854 |
| ence | Equilin | 15 | 238 | 296 | 13 | 225 | 289 |

### EXAMPLES 5-6

### Conjugated Estrogens Tablets

| | mg/Tablet | |
|---|---|---|
| Ingredient | Example 5 | Example 6 |
| Conjugated estrogens‡ | 3.5 | 0.867 |
| Lactose monohydrate | 144 | 87.933 |
| Hydroxypropyl methylcellulose (Methocel K | 54 | 18 |
| 100 M CR Premium) | | |
| Microcrystalline cellulose (Avicel PH101) | 34.6 | — |
| Tricalcium phosphate | — | 12 |
| Meglumine | 1.5 | — |
| Isopropyl alcohol* | 146.31 | 76 |
| Water* | 97.54 | 50.6 |
| Magnesium stearate | 2.4 | 1.2 |
| Opadry Yellow | 9.6 | — |
| Isopropyl alcohol* | 91.2 | — |
| Methylene chloride* | 91.2 | — |

*Evaporates during processing.
‡0.867 mg and 3.5 mg of conjugated estrogens contains 0.252 mg and 1.05 mg of estrone and equilin for Example 6 and Example 5 respectively.

Manufacturing Process for Example 5:

1) Lactose monohydrate, Avicel and methocel K 100 M CR were sifted through an ASTM #40 mesh sieve.

2) Step 1) materials were loaded into a fluidized bed coater and mixed for 5 minutes.

3) Meglumine was dissolved in water and mixed with isopropyl alcohol. This solution was sprayed onto the step 2) materials to form granules.

4) Granules of step 3) were dried at 50 to 55° C. and the granules were passed through an ASTM #60 mesh sieve.

5) Fine particles were separated from step 4) granules by passing through an ASTM #100 mesh sieve.

6) Conjugated estrogens were passed through an ASTM #100 mesh sieve.

7) Fine particles obtained from step 5) and the conjugated estrogens of step

6) were co-sifted through an ASTM #100 mesh sieve in geometric proportion.

8) Coarse granules that were retained on the #100 mesh sieve in step 5) and the material obtained in step 7) were passed through an ASTM #60 mesh sieve and then blended in a blender.

9) Magnesium stearate was sifted through an ASTM #60 mesh sieve and was blended with step 8) materials.

10) Step 9) blend was compressed into tablets.

11) Opadry Yellow, isopropyl alcohol, and methylene chloride were mixed together to prepare a coating mixture.

Manufacturing process for Example 6 is similar to that of Example 5, except that there is no meglumine in step 3) and tribasic calcium phosphate is included in step 6).

The tablets prepared according to Example 5 were subjected to stability testing under the storage conditions of 40° C. and 75% RH for 3 months, in HDPE bottles with molecular sieve desiccant. Analyses were conducted for estrone and equiline content in the conjugated estrogens, and dissolution testing was conducted. Analytical results are shown in Table 6, where values for estrone, equiline and total are percentages of the total conjugated estrogens.

Dissolution conditions were the same as in Examples 2-4.

TABLE 6

| Component | Initial | 3 Months |
|---|---|---|
| Estrone | 56.67 | 57.19 |
| Equiline | 22.26 | 29.01 |
| Total | 78.9 | 86.2 |
| Equilin:Estrone | 0.39 | 0.51 |

Dissolution Testing

| | |
|---|---|
| | Cumulative % of |
| Hours | Drug Dissolved |

| | | |
|---|---|---|
| 1 | 23 | 27 |
| 2 | 36 | 43 |
| 5 | 65 | 71 |
| 8 | 85 | 86 |
| 10 | 92 | 91 |
| 12 | 98 | 92 |

### EXAMPLE 7

### Conjugated Estrogens 0.3 mg Tablets

| Ingredient | mg/Tablet |
|---|---|
| Lactose monohydrate | 87.932 |
| Hydroxypropylmethyl cellulose | 18 |
| (Methocel K 100 M CR Premium) | |
| Tricalcium phosphate | 12 |
| Methanol* | 60 |
| Conjugated estrogens | 0.867 |
| Magnesium stearate | 1.2 |

*Evaporates during processing.

Manufacturing Process:

1) Lactose monohydrate and methocel K 100 M CR were sifted through an ASTM #40 mesh sieve.

2) Tricalcium phosphate was passed through an ASTM #100 mesh sieve and added to the step 1) materials, then loaded into a rapid mixer granulator and mixed for about 5 minutes.

3) Conjugated estrogens were dissolved in methanol to form a drug solution.

4) Step 2) ingredients were granulated using the drug solution

5) Granules obtained from step 4) were dried at 40° C. and passed through an ASTM #60 mesh sieve.

6) Magnesium stearate was sifted through an ASTM #60 mesh sieve and blended with the dried granules.

7) The blend of step 7) was compressed into tablets.

The tablets prepared were subjected to content uniformity testing using the procedure of USP Test 905 “Uniformity of Dosage Units,” and results are shown in Table 8, where the average assay is based on the label conjugated estrogen content.

TABLE 8

| Parameter | Percent |
|---|---|
| Average assay | 96.8 |
| Standard deviation (SD) | 3.01 |
| Relative standard deviation (RSD) | 3.01 |

### EXAMPLES 8-9

### Conjugated Estrogens Pre-Mixes

Grams

| Ingredient | Example 8 | Example 9 |
|---|---|---|
| Conjugated estrogens | 1 | 1 |
| Ethanol* | q.s. | q.s. |
| Water* | q.s. | q.s. |
| Lactose monohydrate | 6.666 | 10 |
| impalpable | | |
| Tricalcium phosphate | 3.333 | — |
| (Calipharm ™ T) @ | | |

*Evaporates during processing.
@ Calipharm T is manufactured by Rhodia and supplied by Signet.

Manufacturing Process:

1) Conjugated estrogens was dissolved in a mixture of ethanol and water.

2) Lactose monohydrate and tricalcium phosphate (if required) were loaded into a fluidized bed processor.

3) Drug solution from step 1) was sprayed onto step 2), followed by drying.

Physical parameters of the pre-mixes are shown below.

| Parameter | Example 8 | Example 9 |
|---|---|---|
| D₉₀, μm | 75 | 75 |
| Bulk density, g/mL | 0.40 | 0.50 |

### EXAMPLE 10

### Conjugated Estrogens Tablets

| | mg/Tablet | | |
|---|---|---|---|
| Ingredient | 10A | 10B | 10C |
| Estrone + Equilin | 0.3 mg | 0.625 mg | 1.25 mg |
| Lactose monohydrate | 54 | 54 | 108 |
| (impalpable) | | | |
| Hypromellose 2208 (Methocel | 18 | 18 | 36 |
| K100 M CR Premium) | | | |
| Isopropyl alcohol* | 51.84 | 51.84 | 103.68 |
| Water* | 34.56 | 34.56 | 69.12 |
| Conjugated estrogens | 0.836 | 1.74 | 3.48 |
| Tribasic calcium phosphate | 12 | 12 | 24 |
| Lactose monohydrate | 27.964 | 27.06 | 54.12 |
| (Pharmatose DCL 11) | | | |
| Hypromellose 2208 (Methocel | 4.8 | 4.8 | 9.6 |
| K100 M CR Premium) | | | |
| Magnesium stearate | 2.4 | 2.4 | 4.8 |

Extended-Release Coating

| | | | |
|---|---|---|---|
| Hypromellose 2910 (Methocel | 14.4 | 14.4 | 43.2 |
| E50 LV) | | | |
| Isopropyl alcohol* | 317.5 | 317.5 | 952.6 |
| Methylene chloride* | 317.5 | 317.5 | 952.6 |
| Water* | 70.1 | 70.1 | 211.68 |

Final Coating

| | | | |
|---|---|---|---|
| Opadry Green @ | 2.69 | — | — |
| Opadry Maroon $ | — | 2.69 | — |
| Opadry Yellow # | — | — | 5.66 |
| Isopropyl alcohol* | 25.56 | 25.56 | 53.81 |
| Methylene chloride* | 25.56 | 25.56 | 53.81 |

*Evaporates during processing.
@ Opadry Green is supplied by Colorcon and contains HPMC 2910/Hypromellose 6 cps, titanium dioxide, D&C Yellow # 10 aluminum lake, FD&C Blue # 2/Indigo Carmine aluminum lake, and macrogol/PEG 400.
$ Opadry Maroon is supplied by Colorcon and contains HPMC 2910/Hypromellose 6 cps, titanium dioxide, FD&C Blue # 2/Indigo Carmine aluminum lake, macrogol/PEG 400, and FD&C Red # 40/Allura Red AC aluminum lake.
# Opadry Yellow is supplied by Colorcon and contains HPMC 2910/Hypromellose 6 cps, titanium dioxide, D&C Yellow # 10 aluminum lake, macrogol/PEG 400, and FD&C Yellow # 6/Sunset Yellow FCF aluminum lake.

Manufacturing Process:

1) Lactose monohydrate impalpable and Methocel K 100 M CR were sifted through an ASTM #40 mesh sieve, loaded into a fluid bed processor, and mixed.

2) The mixture of step 1) was granulated with a mixture of isopropyl alcohol and water, and the granules were dried until less than 2% by weight loss on drying at 105° C. was achieved.

3) The dried granules of step 2) were sifted through an ASTM #60 mesh sieve and were passed through an ASTM #100 mesh sieve.

4) Conjugated estrogens, tricalcium phosphate, and an amount of granules from step 3) equivalent to the sum of required conjugated estrogens and tricalcium phosphate, were blended for about 30 minutes.

5) The step 4) mixture was sifted through an ASTM #100 mesh sieve. Pharmatose DCL 11 and Methocel K 100 M CR (second quantity) were passed through a #60 mesh sieve. These ingredients were mixed with the remaining granules from step 3) in a blender

6) Magnesium stearate was sifted through an ASTM #60 mesh sieve, added to step 5) and blended for 5 minutes.

7) The lubricated blend was compressed into tablets.

8) Tablets of step 7) were coated with Methocel E 50 LV (dissolved in a mixture of isopropyl alcohol, methylene chloride and water).

9) The tablets of step 8 were coated finally with a dispersed Opadry product.

The tablets prepared according to 10C were subjected to in vitro dissolution testing in 900 mL pH 4.5 acetate buffer with sinkers, USP apparatus type II with 50 rpm stirring, and compared with PREMARIN® 1.25 mg tablets. The results are shown in Table 9.

TABLE 9

Cumulative % of
Drug Dissolved

| Hours | 10C | PREMARIN ® |
|---|---|---|
| 1 | 5 | 6 |
| 2 | 21 | 25 |
| 3 | 36 | 41 |
| 4 | 50 | 54 |
| 5 | 60 | 63 |
| 8 | 82 | 84 |
| 10 | 91 | 91 |
| 12 | 96 | 95 |

Tablets prepared as 10C and PREMARIN® 1.25 mg reference tablets were stored under conditions of 40° C. and 75% relative humidity for 3 months in closed HDPE bottles, containing a silica gel desiccant. The estrone and equiline percentages of the conjugated estrogens content, and weight ratios of equiline to estrone in each tablet, were determined at intervals and the data are shown in Table 10.

TABLE 10

| | |
|---|---|
| | PREMARIN ® |
| 10C | 1.25 mg |

| Component | Initial | 3 Mo. | Initial | 3 Mo. |
|---|---|---|---|---|
| Estrone | 56.79 | 56.33 | 55.52 | 53.04 |
| Equiline | 23.11 | 22.7 | 27.37 | 26.21 |
| Total | 79.90 | 79.03 | 82.90 | 79.25 |
| Equiline:Estrone | 0.41 | 0.4 | 0.49 | 0.49 |

### EXAMPLE 11

### Conjugated Estrogens Tablets

| | mg/Tablet | | |
|---|---|---|---|
| Ingredient | 11A | 11B | 11C |
| Estrone + Equilin | 0.3 mg | 0.625 mg | 1.25 mg |
| Conjugated estrogens | 0.87 | 1.817 | 3.63 |
| Tribasic calcium phosphate | 12 | 12 | 24 |
| (Calipharm T) | | | |
| Lactose monohydrate | 83.13 | 82.183 | 164.37 |
| (Pharmatose DCL 11 $) | | | |
| Hypromellose 2208 (Methocel | 21 | 21 | 42 |
| K100 M CR Premium) | | | |
| Silicon dioxide (Syloid 244 | 0.6 | 0.6 | 1.2 |
| FP) | | | |
| Magnesium stearate | 2.4 | 2.4 | 4.8 |

Extended-Release Coating

| | | | |
|---|---|---|---|
| Hypromellose 2910 (Methocel | 16.8 | 16.8 | 40.8 |
| E50 LV) | | | |
| Isopropyl alcohol* | 370.44 | 370.44 | 899.64 |
| Methylene chloride* | 370.44 | 370.44 | 899.64 |
| Water* | 82.32 | 82.32 | 199.92 |

Final Coating

| | | | |
|---|---|---|---|
| Opadry Green or Opadry | 4.10 | 4.10 | 8.42 |
| Maroon or Opadry Yellow | | | |
| Isopropyl alcohol* | 38.99 | 38.99 | 80.03 |
| Methylene chloride* | 38.99 | 38.99 | 80.03 |

*Evaporates during processing.
$ Supplied by DMV.

Manufacturing Process:

1) Lactose monohydrate was passed through an ASTM #100 mesh sieve, preparing a coarse fraction and a fine fraction in a weight ratio of about 1:9.

2) Conjugated Estrogens, tricalcium phosphate and an equivalent weight of fine fraction lactose monohydrate from step 1) were loaded in a blender and the mixture was blended for about 5 minutes.

3) Mixture of step 2) was passed through an ASTM #100 mesh sieve blended with an equivalent weight of fine fraction lactose monohydrate from step 1) for about 5 minutes.

4) Coarse fraction lactose monohydrate, Methocel K 100 M CR and Syloid 244 FP were sifted through an ASTM #60 mesh sieve.

5) The mixture of step 3), the mixture of step 4), and remaining fine fraction lactose monohydrate were loaded into a blender and blended for 30 minutes.

6) Magnesium stearate was passed through an ASTM #60 mesh sieve and blended with the mixture of step 5) for 5 minutes.

7) The mixture of step 6) was compressed into tablets.

8) Tablets of step 7) were coated with Methocel E 50 LV (dissolved in a mixture of isopropyl alcohol, methylene chloride and water) followed by a final coating with an Opadry dispersion.

The tablets prepared according to 11C were subjected to in vitro dissolution testing in 900 mL of pH 4.5 acetate buffer with sinkers, USP apparatus type II with 50 rpm stirring, and compared with PREMARIN® 1.25 mg tablets. The results are shown in Table 11.

TABLE 11

Cumulative % of
Drug Dissolved

| Hours | 11C | PREMARIN ® |
|---|---|---|
| 1 | 6 | 6 |
| 2 | 24 | 25 |
| 3 | 42 | 41 |
| 4 | 57 | 54 |
| 5 | 69 | 63 |
| 8 | 91 | 84 |
| 10 | 98 | 91 |
| 12 | 103 | 95 |

Tablets prepared in 11C and PREMARIN® 1.25 mg reference tablets were stored under conditions of 40° C. and 75% RH for 3 months in closed HDPE bottles, containing a silica gel desiccant. The estrone and equiline percentages of the conjugated estrogens content, and weight ratios of equiline to estrone in each tablet, were determined at intervals and the data are shown in Table 12.

TABLE 12

| | |
|---|---|
| | PREMARIN ® |
| 11C | 1.25 mg |

| Component | Initial | 3 Mo. | Initial | 3 Mo. |
|---|---|---|---|---|
| Estrone | 60.71 | 63.94 | 59.52 | 57.61 |
| Equilin | 25.11 | 24.92 | 27.01 | 26.97 |
| Total | 85.8 | 88.9 | 86.5 | 84.6 |
| Equiline:Estrone | 0.41 | 0.39 | 0.45 | 0.47 |

We claim:

1. A pre-mix composition, prepared by spray drying or spray coating a solution comprising conjugated estrogens, or a dispersion comprising conjugated estrogens in solution and a solid pharmaceutical carrier, or by spray coating a solution or dispersion comprising conjugated estrogens onto a solid pharmaceutical carrier.

2. The premix composition of claim 1, wherein a solution or dispersion comprises an organic solvent.

3. The premix composition of claim 1, wherein a pharmaceutical carrier comprises one or more of lactose, dextrose, mannitol, and sorbitol.

4. The premix composition of claim 1, wherein a pharmaceutical carrier comprises lactose.

5. The premix composition of claim 1, wherein a pharmaceutical carrier comprises an inorganic compound.

6. The premix composition of claim 1, having a moisture content not exceeding about 4 percent by weight.

7. The pre-mix composition of claim 1, wherein the concentration of conjugated estrogens is at least about 0.1 percent by weight of the total composition.

8. The premix composition of claim 1, in which 90 percent of particles have sizes about 20 μm to 150 μm.

9. The premix composition of claim 1, having bulk density about 0.2 to 0.6 grams per mL.

10. A pharmaceutical formulation comprising a pre-mix composition of claim 1.

11. The pharmaceutical formulation of claim 10, having a moisture content not exceeding about 6 percent by weight.

12. The pharmaceutical formulation of claim 10, providing, following immersion of a single unit dosage form into pH 4.5 acetate buffer, when tested in USP Apparatus II with 50 rpm stirring:
a) release of about 2 to about 30 percent of contained conjugated estrogens within about one hour;
b) release of about 5 to about 55 percent of contained conjugated estrogens within about 2 hours;
c) release of about 60 to about 100 percent of contained conjugated estrogens within about 5 hours; and
d) release of not less than about 70 percent of contained conjugated estrogens within about 8 hours.

13. The pharmaceutical formulation of claim 10, providing, following immersion of a single unit dosage form into degassed purified water, when tested in USP Apparatus II with 50 rpm stirring:
a) release of less than about 35 percent of contained conjugated estrogens within about one hour;
b) release of less than about 65 percent of contained conjugated estrogens within about 2 hours;
c) release of about 30 to about 100 percent of contained conjugated estrogens within about 5 hours; and
d) release of not less than about 60 percent of contained conjugated estrogens within about 8 hours.

14. The pharmaceutical formulation of claim 10, containing 0.625 mg of conjugated estrogen and producing: estrone Cₘₐₓ values about 58 pg/mL to about 90 pg/mL, AUC₀₋ₜ values about 2340 pg·hour/mL to about 3658 pg·hour/mL, and AUC_{0-∞} values about 5754 pg·hour/mL to about 8991 pg·hour/mL; and equilin Cₘₐₓ values about 10 pg/mL to about 16 pg/mL, AUC₀₋ₜ values about 187 pg·hour/mL to about 292 pg·hour/mL, and AUC_{0-∞} values about 242 pg·hour/mL to about 378 pg·hour/mL; in plasma after oral administration of a single dose to healthy humans under fasting conditions.

15. The pharmaceutical formulation of claim 10, containing 0.625 mg of conjugated estrogen and producing: estrone Cₘₐₓ values about 57 pg/mL to about 89 pg/mL, AUC₀₋ₜ values about 1903 pg·hour/mL to about 2974 pg·hour/mL, and AUC_{0-∞} values about 2989 pg·hour/mL to about 4670 pg·hour/mL; and equilin Cₘₐₓ values about 11 pg/mL to about 18 pg/mL, AUC₀₋ₜ values about 174 pg·hour/mL to about 272 pg·hour/mL, and AUC_{0-∞} values about 216 pg·hour/mL to about 338 pg·hour/mL; in plasma after oral administration of a single dose to healthy humans under fed conditions.

16. The pharmaceutical formulation of claim 10, providing Cₘₐₓ and AUC₀₋ₜ values that do not vary by more than about 20% between fed state administration and fasted state administration, to healthy humans.

17. A process for preparing a pre-mix composition, comprising spray drying a dispersion comprising a solution of conjugated estrogens in solution and a solid pharmaceutical carrier.

18. The process of claim 17, wherein a solution comprises an organic solvent.

19. A process for preparing a pre-mix composition, comprising spray coating a solution or dispersion comprising conjugated estrogens onto a solid pharmaceutical carrier.

20. The process of claim 19, wherein a solution or dispersion comprises an organic solvent.

21. The process of claim 19, wherein 90 percent of particles in the premix composition have sizes about 20 μm to 150 μm.
